(19) United States
(12) Patent Application Publication
(10) Pub. No.: US 2022/0235373 A1
(43) Pub. Date: Jul. 28, 2022

(54) AGENT THAT ENABLES SIRT7 GENE EXPRESSION AND THE USE THEREOF

(71) Applicant: SHENZHEN UNIVERSITY, Shenzhen, Guangdong (CN)

(72) Inventors: Baohua LIU, Shenzhen, Guangdong (CN); Shimin SUN, Shenzhen, Guangdong (CN); Xiaolong TANG, Shenzhen, Guangdong (CN)

(73) Assignee: SHENZHEN UNIVERSITY, Shenzhen, Guangdong (CN)

(21) Appl. No.: 17/610,637

(22) PCT Filed: Oct. 12, 2019
(86) PCT No.: PCT/CN2019/110813

§ 371 Date: Nov. 11, 2021

(51) Int. Cl. C12N 15/86

(52) U.S. Cl. CPC C12N 15/86; C12N 2800/30; C12N 2750/14143

(57) ABSTRACT

The present invention provides an agent that enables Sirt7 gene expression, especially a recombinant adeno-associated virus (rAAV) that enables vascular endothelium (VE)-specific Sirt7 gene expression, and the use thereof. The present invention also provides a method for improving neovascularization, ameliorating aging features, extending lifespan and treating age-related diseases by using the agent, especially the rAAV.

This application is the U.S. national phase of PCT Application No. PCT/CN2019/110813 filed on Oct. 12, 2019, the disclosure of which is incorporated in its entirety by reference herein.

### TECHNICAL FIELD

The present invention relates to gene target therapy. In particular, the present invention relates to an agent that enables Sirt7 gene expression, which is useful in rejuvenating blood vessels, extending lifespan and treating age-related diseases.

### BACKGROUND

Aging represents the largest risk factor for many age-related diseases, as exemplified by cardiovascular diseases (CVDs) (1). The blood vessel consists of the tunica intima (composed of endothelial cells; ECs), the tunica media (composed of vascular smooth muscle cells; VSMCs) and the tunica adventitia (consisting of connective tissue) (2). The endothelium separates the vessel wall from blood flow and has an irreplaceable role in regulating vascular tone and homeostasis (3, 4). Age-related functional decline in ECs and VSMCs is a main cause of CVDs (4-6). ECs secrete various vasodilators and vasoconstrictors that act on VSMCs and induce blood-vessel contraction and relaxation (7). For instance, nitric oxide (NO) is synthesized from L-arginine by endothelial NO synthase (eNOS) and then released on VSMCs to induce blood-vessel relaxation (8). When ECs become senescent or dysfunctional, vasoconstrictive, pro-coagulative and pro-inflammatory cytokines are released; this effect reduces NO bioavailability and in turn increases vascular intimal permeability and EC migration (9). Despite advances in the understanding of mechanisms of endothelial dysfunction, it is unclear whether it directly triggers organismal aging.

Accumulating evidences suggest that the mechanisms underlying physiological aging are similar to those governing Hutchinson-Gilford progeria syndrome (HGPS)—a premature aging syndrome in which affected patients typically succumb to CVDs (10-15). HGPS is predominantly caused by an a.c. 1824 C>T, p. G608G mutation in LMNA gene, which activates an alternate splicing event and generates a 50-amino-acid-truncated form of lamin A, referred to as progerin (10). The murine Lmna^{G609G}, which is equivalent to LAMA^{G608G} in humans, causes aging phenotypes resembling HGPS (16). It has been shown that progerin targets SMCs and causes blood vessel calcification and atherosclerosis (17-22). Recent work by two groups showed that SMC-specific progerin knock-in mice are healthy and have a normal lifespan, but suffer from blood-vessel calcification, atherosclerosis and shortened lifespan when crossed to Apoe^{-/-} mice (23, 24). In contrast to SMCs, the contributing roles of the vascular endothelium (VE) to systemic/organismal aging are still elusive. It is still urgent to investigate the roles of VE dysfunction to systemic aging and the targeting potential for the clinical treatment of HGPS.

### SUMMARY OF THE INVENTION

We generated a knock-in mouse model with the causative HGPS Lmna^{G609G} mutation, called progerin. We crossed Lmna^{f/f} mice with a Tie2-Cre line to get Lmna^{f/f}; TC mice, which exhibit defective microvasculature and neovascularization, accelerated aging and shortened lifespan. Single-cell transcriptomic analysis of murine lung endothelial cells (MLECs) revealed a significant upregulation of inflammatory response. At molecular level, progerin interacts and destabilizes NAD⁺-dependent deacylase Sirt7; ectopic expression of Sirt7 alleviates the inflammatory response caused by progerin in HUVECs. Most remarkably, vascular endothelium-targeted Sirt7 gene therapy, driven by a ICAM2 promoter in an rAAV1 vector, improves neovascularization, ameliorates aging features and extends lifespan by more than 75% in Lmna^{f/f}; TC mice. These data support endothelial dysfunction as a primary trigger of systemic aging and highlight gene therapy as a potential strategy for the clinical treatment of HGPS and age-related vascular dysfunction.

In the first aspect, the present invention provides an agent that enables the Sirt7 gene expression.

In the second aspect, the present invention provides use of the agent according to the first aspect in the manufacture of a medicament for improving neovascularization, ameliorating aging features, preventing aging, extending lifespan, and/or treating Hutchinson-Gilford progeria syndrome (HGPS) and/or age-related diseases.

In the third aspect, the present invention provides a method for improving neovascularization, ameliorating aging features, preventing aging, extending lifespan, and/or treating Hutchinson-Gilford progeria syndrome (HGPS) and/or age-related diseases, comprising administering a pharmaceutically effective amount of the agent according to the first aspect to a subject in need thereof.

### DESCRIPTION OF THE FIGURES

FIGS. 1A-1D show single-cell transcriptomic profiles of CD31⁺ MLECs. (FIG. 1A) Purity analysis of sorted CD31⁺ MLECs by FACS. (FIG. 1B) t-SNE projection of CD31⁺ cells revealed four clusters: endothelial cells (ECs), B lymphocytes (B-like), T lymphocytes (T-like) and Macrophages (Mϕ-like). (FIG. 1C) Marker gene expression in the four clusters: ECs (Cd31, Cd34, Cdh5), B-like (Ly6d, Cd22, Cd81), T-like (Cd3d, Cd3e, Cd28) and Mϕ-like (Cd14, Cd68, Cd282). (FIG. 1D) Heatmap showing marker gene expression levels in Lmna^{G609G/G609G} (G609G) and Lmna^{f/f} (Flox) mice.

FIG. 2A-2E show that single-cell transcriptomic analysis indicates an inflammatory response and cardiac dysfunction in progeroid ECs. (FIG. 2A) t-SNE projection of Lmna^{G609G/G609G} (G609G, green) and Lmna^{f/f} (Flox, orange) CD31⁺ MLECs, according to transcriptomic data. (FIGS. 2B-D) GO and KEGG pathway enrichment of differentially expressed genes between G609G and Flox cells. Lmna^{G609G/G609G} MLECs show enrichment in genes that regulate the inflammatory response (FIG. 2C) and genes related to heart dysfunction (FIG. 2D). (FIG. 2E) Quantitative PCR analysis of altered genes observed in (FIG. 2C) and (FIG. 2D) in human umbilical vein endothelial cells (HUVECs) with ectopic expression of progerin or wild type LMNA. Data represent the means±s.e.m. *P<0.05, *P<0.01, *P<0.001 (Student's t test).

FIGS. 3A-D show endothelial-specific dysfunction in progeria mice. (A, B) H&E staining of thoracic aorta sections from (FIG. 3A) Lmna^{f/f}; TC and (FIG. 3B) Lmna^{G609G/G609G} and Lmna^{f/f} control mice showing intima-media thickening. Scale bar, 20 μm. (FIG. 3C) Acetylcholine (Ach)-induced thoracic aorta vasodilation in Lmna^{f/f}; TC and Lmna_{f/f} control mice. **P<0.01. (FIG. 3D) Ach-induced thoracic aorta vasodilation in Lmna^{G609G/G609G} and control mice. **P<0.01. (FIG. 3E) Sodium nitroprusside (SNP)-induced thoracic aorta vasodilation in Lmna^{G609G/G609G} and control mice. (F) eNOS level in thoracic aorta sections from Lmna^{f/f}; TC and control mice. Scale bar, 20 μm. All data represent the means±s.e.m. P values were calculated by Student's t test.

FIG. 4A-D show reduced capillary density and defective neovascularization.

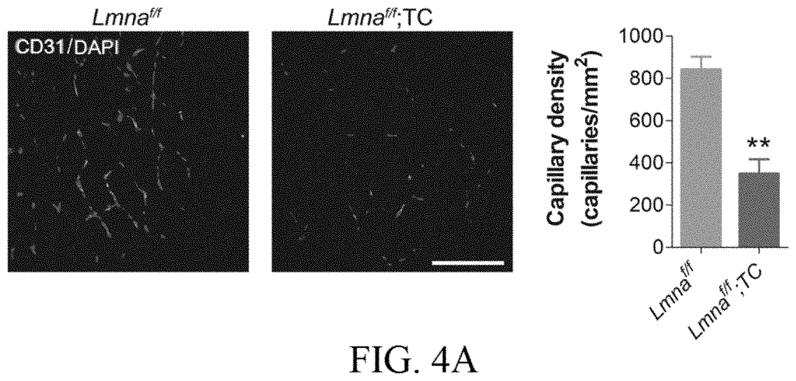

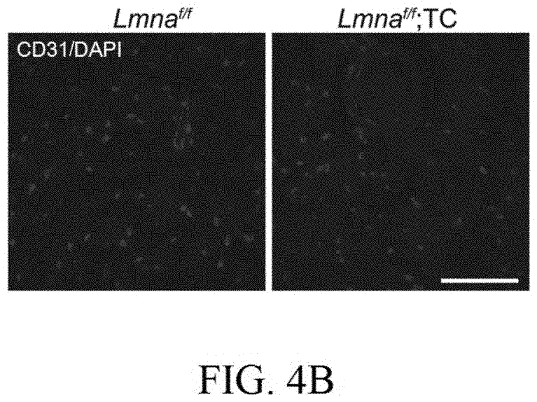

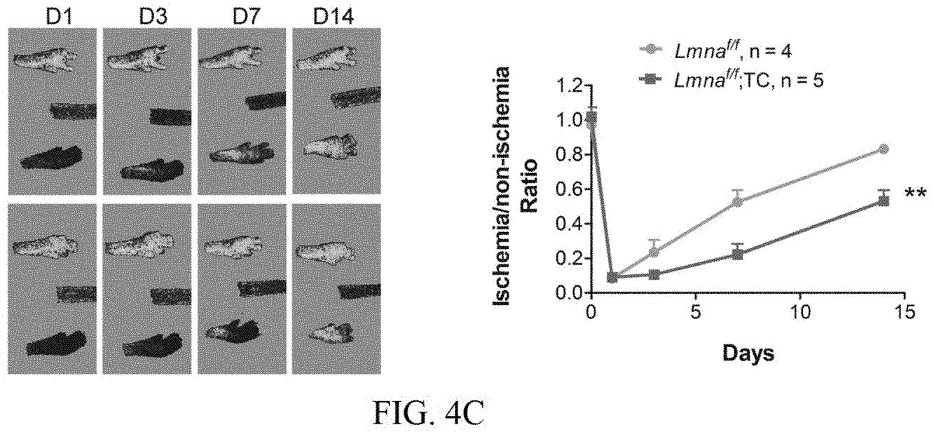

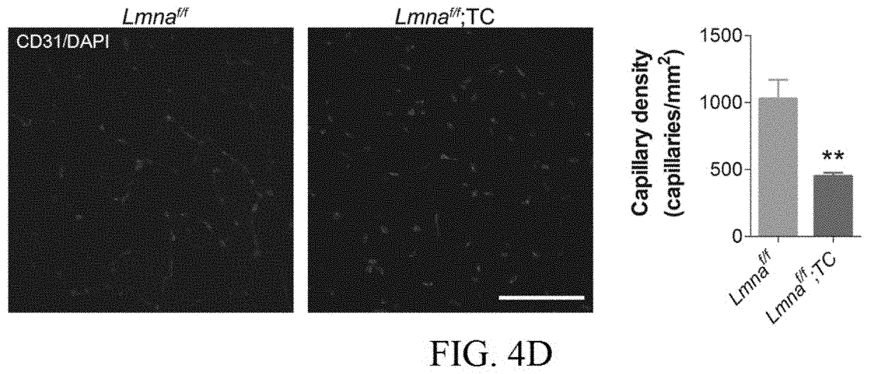

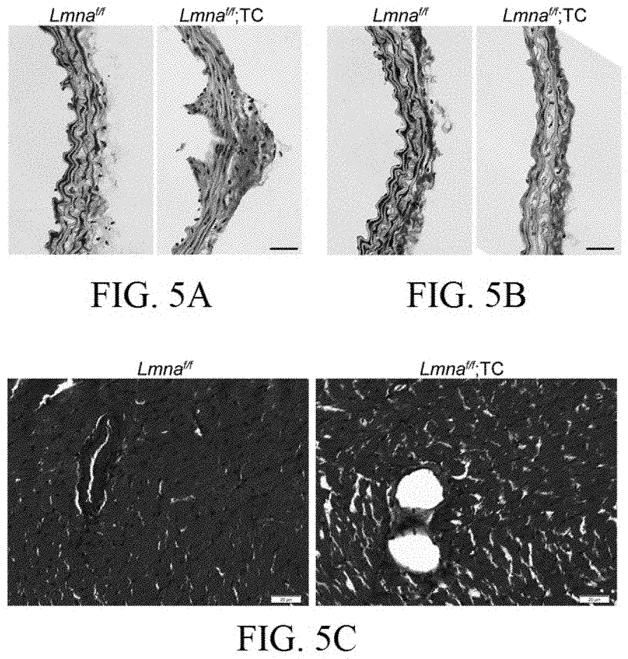

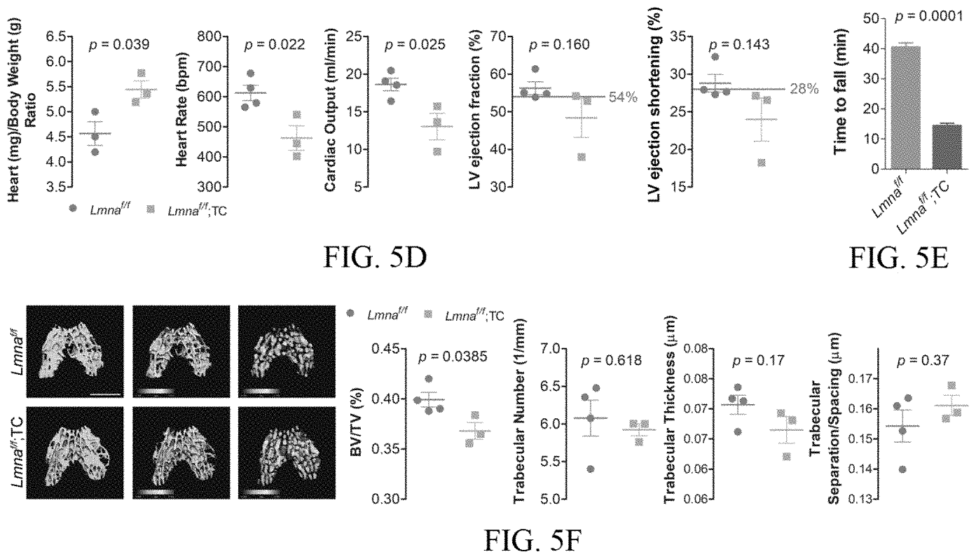

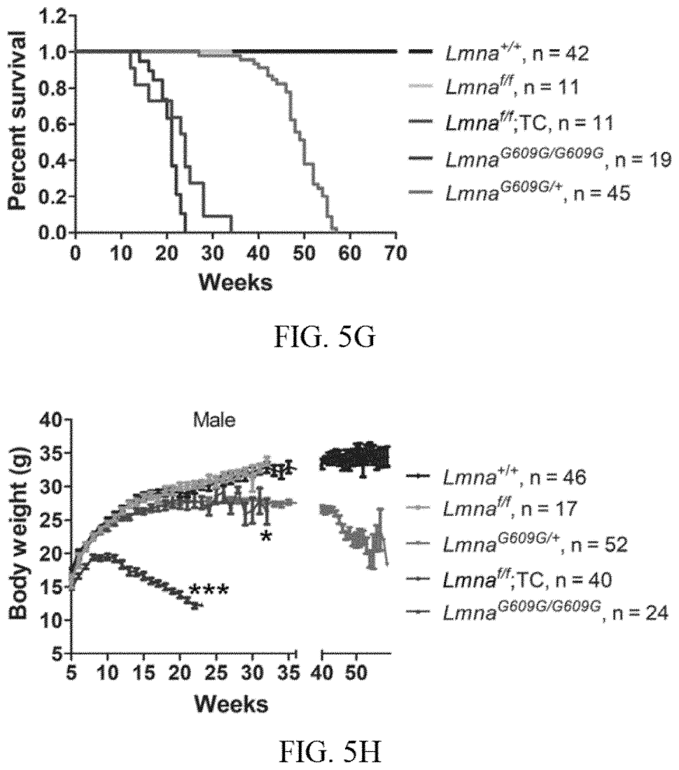

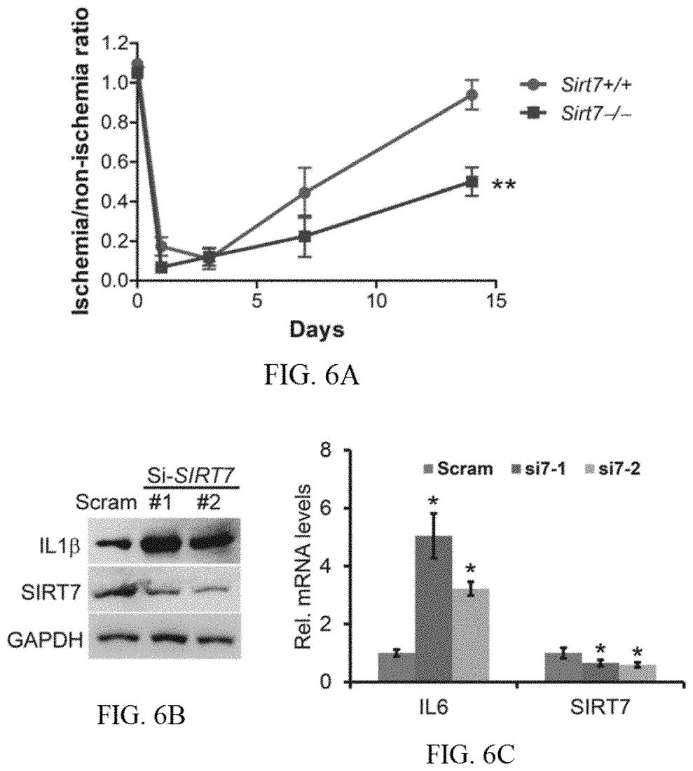

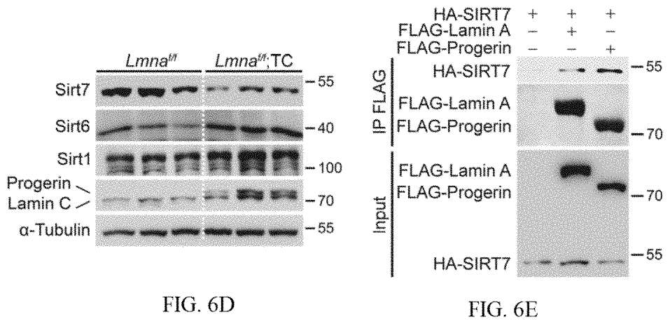

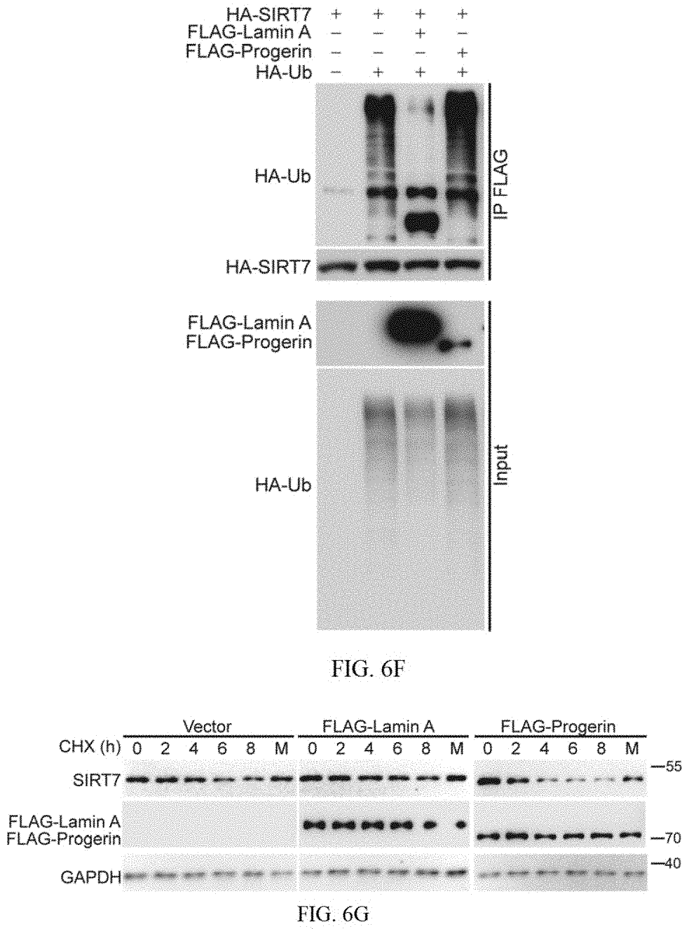

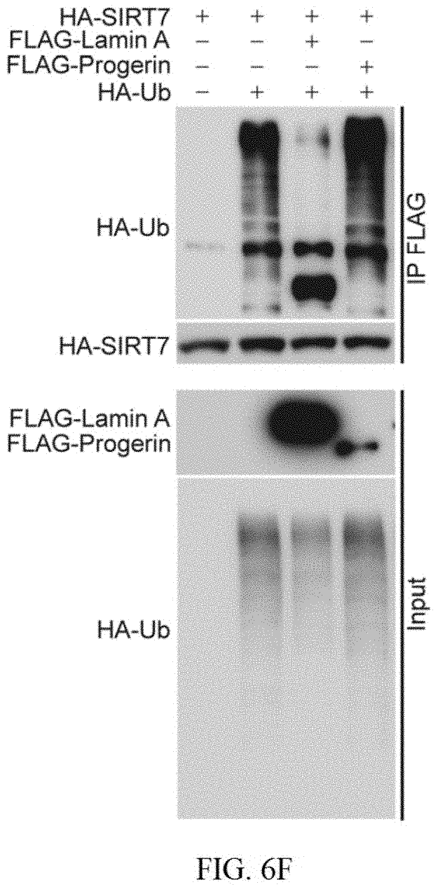

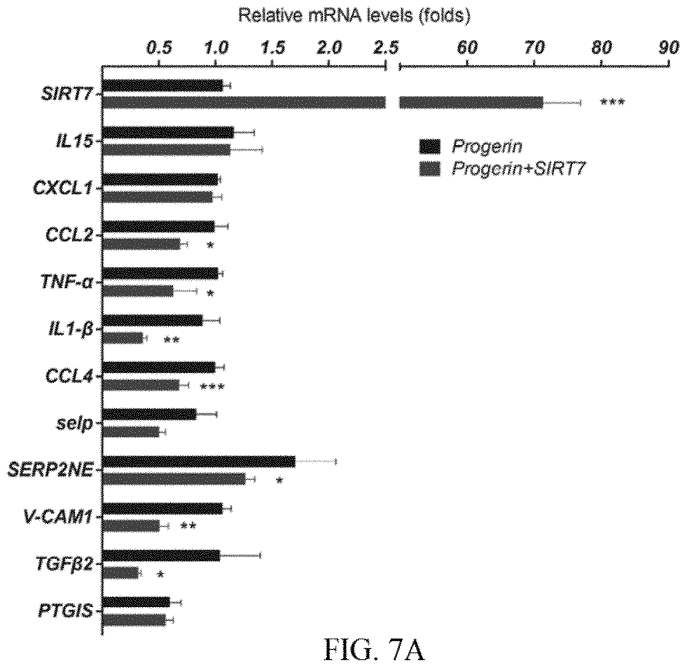

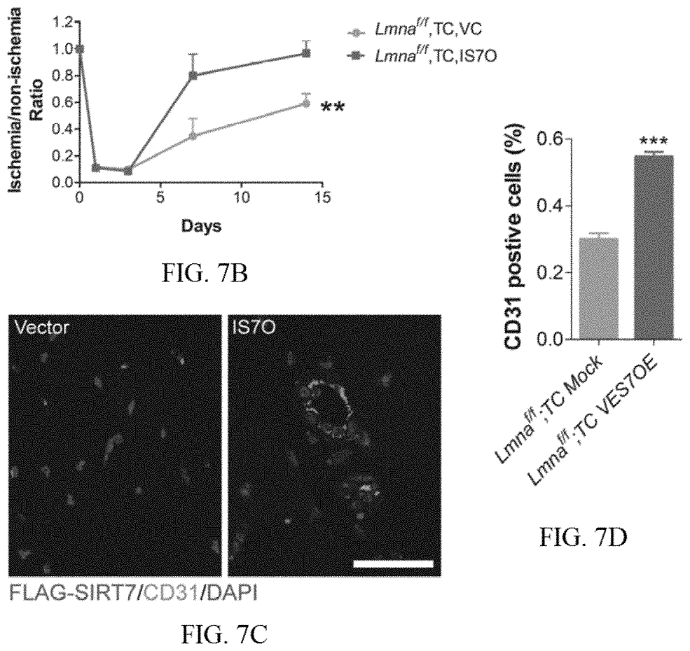

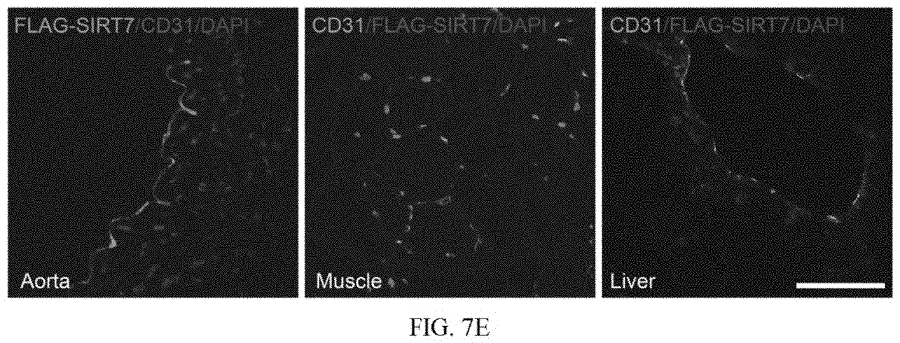

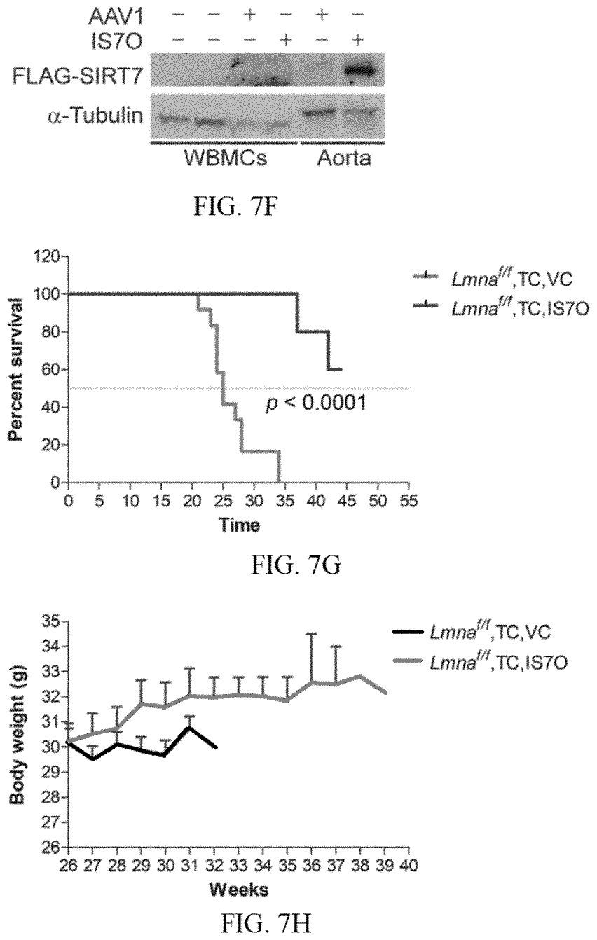

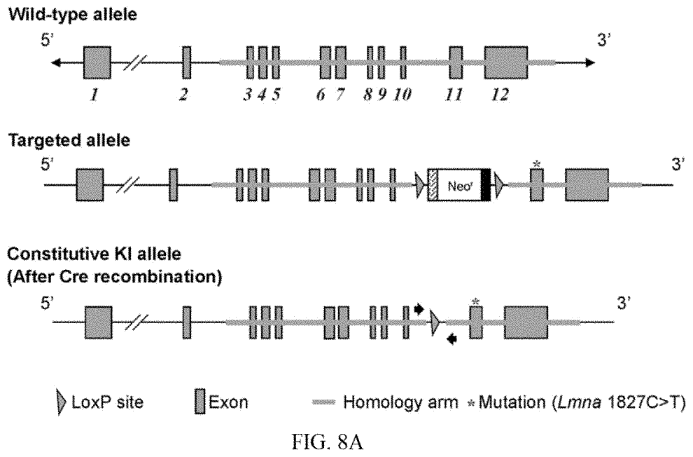

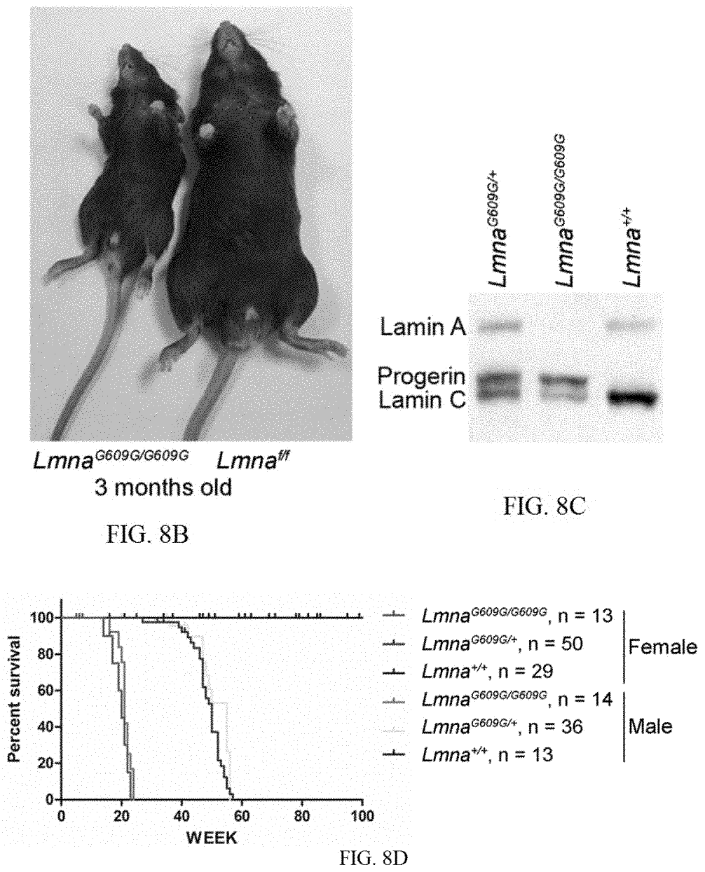

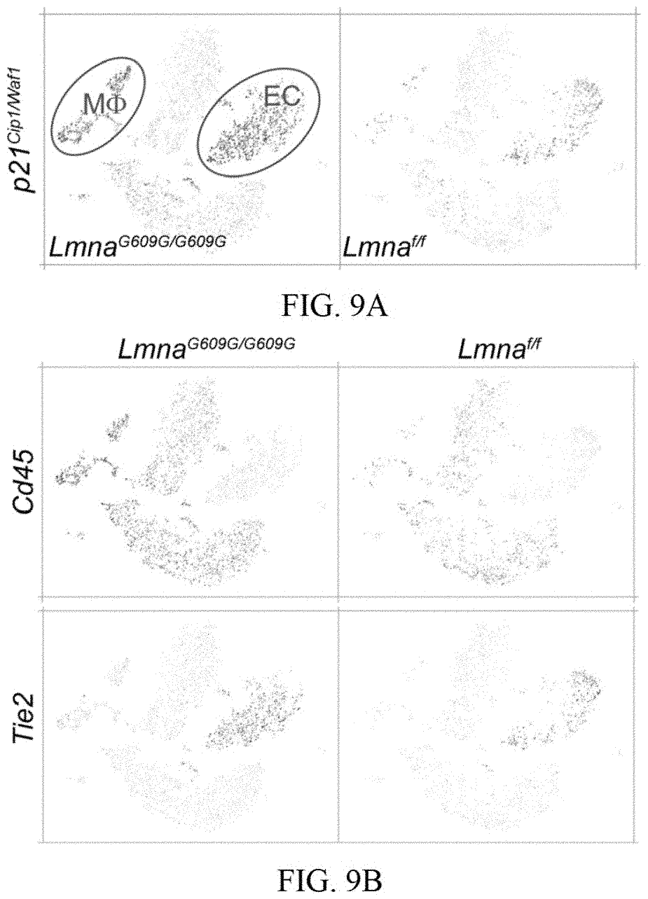

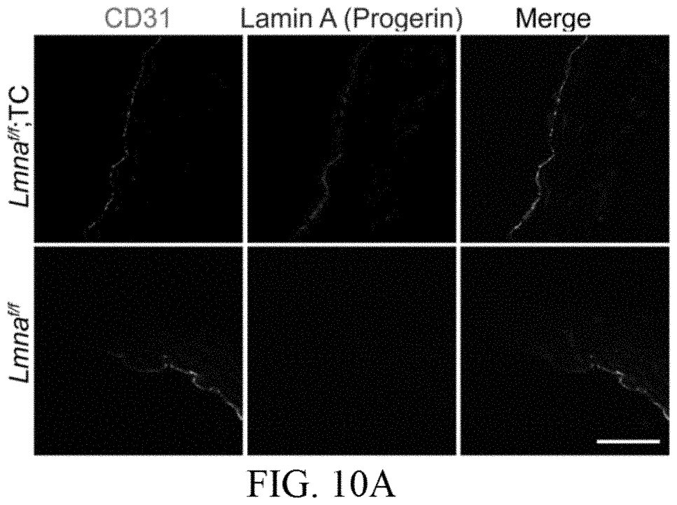

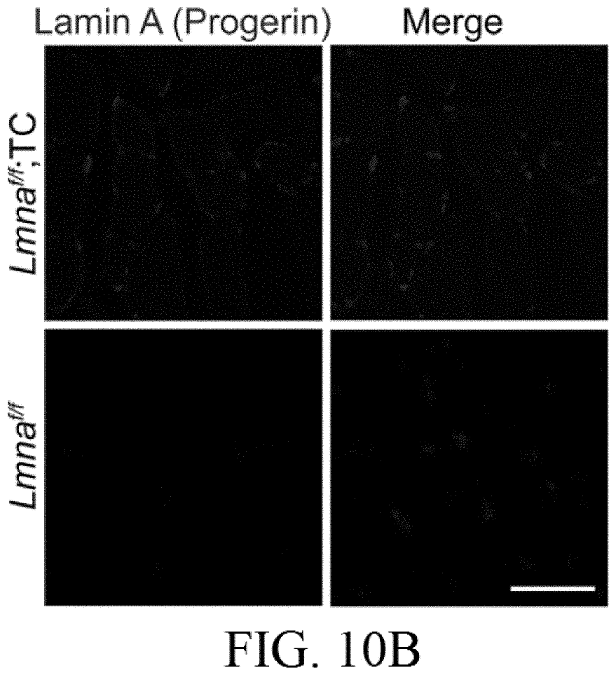

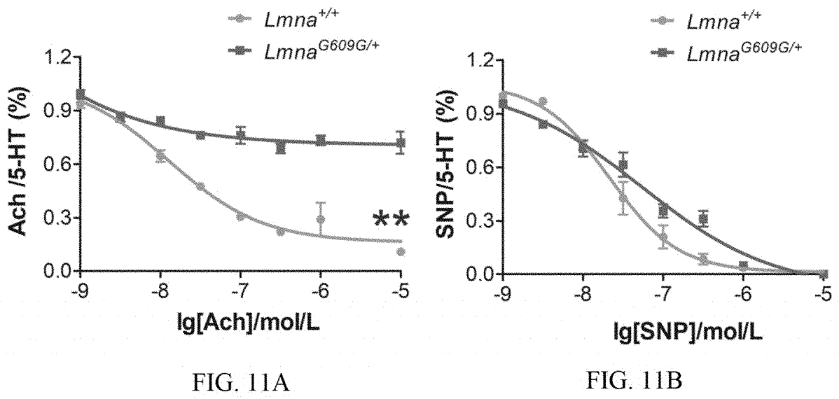

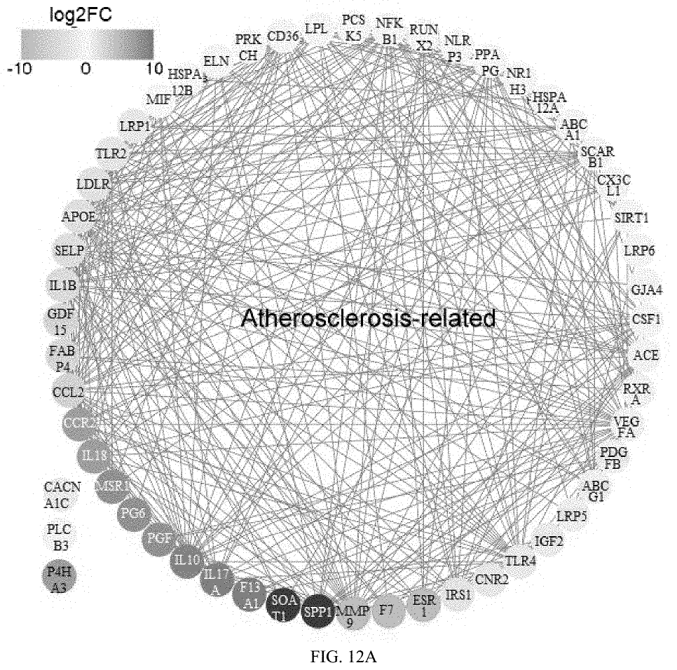

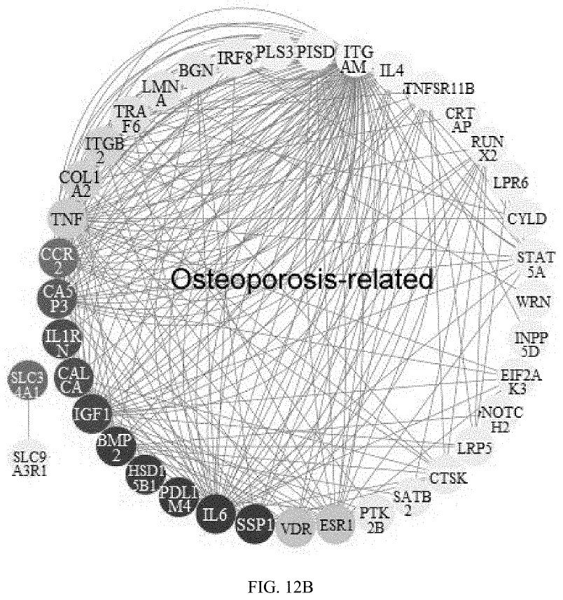

(FIG. 4A) Immunofluorescence staining (left) and quantification (right) of CD31⁺ gastrocnemius muscle in Lmna^{f/f}; TC and Lmna^{f/f} mice. Scale bar, 50 μm. (FIG. 4B) CD31 immunofluorescent staining in Lmna^{f/f}; TC and Lmna^{f/f} liver. Scale bar, 50 μm. (FIG. 4C) Representative microcirculation images (left) and quantification of blood flow recovery (right) following hind limb ischemia in Lmna^{f/f}; TC and Lmna^{f/f} mice. (FIG. 4D) Representative transverse sections and quantification of CD31⁺ gastrocnemius muscle 14 days after femoral artery ligation. Scale bar, 50 μm. All data represent the means±s.e.m. P values were calculated by Student's t test.

FIGS. 5A-H show systemic aging phenotypes in Lmna^{f/f}; TC mice. (FIGS. 5A-C) Masson trichrome staining showing an atheromatous plaque in the aorta (FIG. 5A), smooth muscle cell loss (FIG. 5B) and cardiac fibrosis (FIG. 5C) in Lmna^{f/f}; TC mice. Scale bar, 20 μm. (FIG. 5D) Heart weight and echocardiographic parameters, including heart rate, cardiac output, left ventricular (LV) ejection fraction and LV ejection shortening. *P<0.05, Lmna^{f/f}; TC Vs Lmna^{f/f} mice. (FIG. 5E) Decreased running endurance in Lmna^{f/f}; TC mice. ***P<0.001. (FIG. 5F) Micro-CT analysis showing a decrease in trabecular bone volume/tissue volume (BV/TV), trabecular number (Tb.N) and trabecular thickness (Tb.Th), and an increase in trabecular separation (Tb.Sp) in Lmna^{f/f}; TC mice. *P<0.05, Lmna^{f/f}; TC Vs Lmna^{f/f} mice. (FIG. 5G) Lifespan of Lmna^{G609G/G609G}, Lmna^{G609G/+}, Lmna^{f/f}; TC and Lmna^{f/f} mice. (FIG. 5H) Body weight of male Lmna^{G609G/G609G}, Lmna^{G609G/+}, Lmna^{f/f}; TC and Lmna^{f/f} mice. *P<0.05, Lmna^{f/f}; TC Vs Lmna^{f/f} mice; ***P<0.001, Lmna^{G609G/G609G} Vs Lmna^{f/f} mice. All data represent the means±s.e.m. P values were calculated by Student's t test, except that statistical comparison of the survival data was performed by Log-rank Test.

FIGS. 6A-G show that accumulation of progerin destabilizes Sirt7. (FIG. 6A) Quantification of blood flow recovery following hind limb ischemia in Sirt7^{-/-} and Sirt7^{+/+} mice. (FIG. 6B) Representative immunoblots showing indicated protein levels in HUVECs treated with si-SIRT7 or scramble (Scram). (FIG. 6C) Real-time PCR analysis of indicated gene expression in HUVECs treated with si-SIRT7 or Scram. *P<0.05, siRNA Vs Scram. (FIG. 6D) Representative immunoblots showing indicated sirtuin (Sirt1, Sirt6 and Sirt7) protein levels in FACS-sorted mouse lung endothelial cells (MLECs). Noted downregulated Sirt7 but rather upregulated Sirt6 and hardly changed SIRT1 in Lmna^{f/f}; TC MLECs. (FIG. 6E) Co-immunoprecipitation (Co-IP) experiments showing HA-SIRT7 in anti-FLAG-lamin A and anti-FLAG-progerin immunoprecipitates. (FIG. 6F) Representative immunoblots showing poly-ubiquitinated SIRT7, which was upregulated in presence of progerin but rather downregulated in presence of lamin A. (FIG. 6G) Representative immunoblots showing SIRT7 protein levels in presence of lamin A or progerin in HEK293 cells treated with Cycloheximide (CHX) and/or MG132 (M). All data represent the means±s.e.m. P values were calculated by Student's t test.

FIGS. 7A-H show that VE-targeted Sirt7 therapy rejuvenates the microvasculature and extends lifespan in progeria mice. (FIG. 7A) Real-time PCR analysis of genes that are aberrantly upregulated in progerin-overexpressing HUVECs upon overexpression of SIRT7. *P<0.05, **P<0.01, ***P<0.001. (FIG. 7B) Neovascularization assay in Lmna^{f/f}; TC mice with hind limb ischemia, treated with/without IS7O particles. *P<0.05. (FIG. 7C) Immunofluorescence microscopy analysis of FLAG-SIRT7 and CD31 expression in gastrocnemius muscle 14 days after femoral artery ligation. Scale bar, 25 μm. (FIG. 7D) Percent CD31⁺ ECs in Lmna^{f/f}; TC mice treated with/without IS7O particles. ***P<0.001. (FIG. 7E) Representative immunofluorescence images of the liver, aorta and muscle of Lmna^{f/f}; TC mice after IS7O therapy, showing CD31+ ECs with FLAG-SIRT7 expression. Scale bar, 50 μm. (FIG. 7F) Representative immunoblots showing expression of FLAG-SIRT7 in aorta and whole bone marrow cells (WBMCs). Noted that FLAG-SIRT7 was merely detected in WBMCs. (FIG. 7G) Lifespan of IS70-treated and untreated Lmna^{f/f}; TC and Lmna^{G609G/+} mice. (FIG. 7H) Body weight of IS7O-treated and untreated Lmna^{f/f}; TC and Lmna^{f/f} mice. All data represent the means±s.e.m. P values were calculated by Student's t test, except that the statistical comparison of survival data was performed by Log-rank Test.

FIGS. 8A-D show generation of Lmna^{f/f} mice and phenotypic analysis of Lmna^{G609G/G609G} mice. (FIG. 8A) Schematic illustration of knock-in strategy of Lmna^{f/f} mice harboring Lmna^{G609G} mutation (Lmna 1827C>T); (FIG. 8B) Representative photo of Lmna^{G609G/G609G} mice and Lmna^{f/f} control mice. (FIG. 8C) Representative immunoblot showing Lamin A, Progerin and Lamin A expression in Lmna^{G609G}, Lmna^{G609G/G609G} and Lmna^{+/+} control mice. (FIG. 8D) Lifespan determination of Lmna^{G609G/+}, Lmna^{G609G/G609G} and Lmna^{+/+} mice.

FIGS. 9A-B show single cell transcriptomic analysis of CD31⁺ MLECs. (FIG. 9A) p21^{Cip/Waf1} mRNA levels in Lmna^{G609G/G609G} (G609G) and Lmna^{f/f} (Flox) CD31⁺ MLECs. p21^{Cip/Waf1} was elevated specifically in ECs and MΦ-like cells isolated from G609G mice. (FIG. 9B) Cd45 and Tie2 levels in G609G and Flox CD31⁺ MLECs. Cd45 expression was lacking in ECs and Tie2 expression was EC-specific.

FIGS. 10A-B show VE-specific progerin expression. (FIGS. 10A-B) Progerin and CD31 expression was detected by immunofluorescence staining in aorta (FIG. 10A) and muscle (FIG. 10B) tissue of Lmna^{f/f}; TC and Lmna^{f/f} mice. Scale bar, 50 μm.

FIGS. 11A-B shows vasodilation analysis of Lmna^{G609G/+} mice. Acetylcholine (ACh)-induced (FIG. 11A) and sodium nitroprusside (SNP)-induced (FIG. 11B) vasodilation in Lmna^{G609G/+} and Lmna^{+/+} control mice. **P<0.01.

FIGS. 12A-B shows the expression of atherosclerosis-associated (FIG. 12A) and osteoporosis-associated (FIG. 12B) genes in MLEC transcriptomes.

### PARTICULAR EMBODIMENTS

The present invention provides an agent that enables the Sirt7 gene expression; preferably, the agent is a vector; more preferably, the vector is a plasmid, and/or a virus vector; most preferably, the virus vector is a recombinant adeno-associated virus (rAAV), particularly rAAV serotype 1.

In a specific embodiment, the Sirt7 gene expression is vascular endothelium (VE)-specific expression; particularly, the Sirt7 gene expression is driven by an ICAM2 promoter.

The present invention also provides use of the agent in the manufacture of a medicament for improving neovascularization, ameliorating aging features, preventing aging, extending lifespan, and/or treating Hutchinson-Gilford progeria syndrome (HGPS) and/or age-related diseases. Preferably, the age-related diseases are cardiovascular diseases (CADs), arthritis, amyotrophy, and/or osteoporosis. More preferably, the cardiovascular diseases are heart failure and/or atherosclerosis.

In a specific embodiment, the aging, HGPS, and/or age-related diseases are characterized by vascular endothelium (VE) dysfunction.

The present invention also provides a method for improving neovascularization, ameliorating aging features, preventing aging, extending lifespan, and/or treating Hutchinson-Gilford progeria syndrome (HGPS) and/or age-related diseases, comprising administering a pharmaceutically effective amount of the agent to a subject in need thereof. Preferably, the age-related diseases are cardiovascular diseases (CADs), arthritis, amyotrophy, and/or osteoporosis. More preferably, the cardiovascular diseases are heart failure and/or atherosclerosis.

In a specific embodiment, the aging, HGPS, and/or age-related diseases are characterized by vascular endothelium (VE) dysfunction.

The present invention will be further demonstrated by the following experimental procedures and examples, which are used only for illustration purpose, but not limiting the scope of the present invention.

Experimental Procedures

Animals

Lmna^{f/+} allele (Lmna^{G609G} mutation flanked by 2 loxP sites) was generated by Cyagen Biosciences Inc., China. Briefly, the 5′ and 3′ homology arms were amplified from BAC clone RP23-21K15 and RP23-174J9, respectively. The G609G (GGC to GGT) mutation was introduced into exon 11 in the 3′ homology arm. C57BL/6 embryonic stem cells were used for gene targeting. To obtain ubiquitous expression of progerin (Lmna^{G609G/G609G}), Lmna^{f/f} mice were bred with E2A-Cre mice. To obtain VE-specific expression of progerin, Lmna^{f/f} mice were bred with Tie2-cre mice. Mice were housed and handled in accordance with protocols approved by the Committee on the Use of Live Animals in Teaching and Research of Shenzhen University, China.

Hind Limb Ischemia

Four months old male mice were anesthetized with 4% chloral hydrate (0.20 ml/20 g) by intraperitoneal injection. Hind limb ischemia was performed by unilateral femoral artery ligation and excision, as previously described (48). In brief, the neurovascular pedicle was visualized under a light microscope following a 1-cm incision in the skin of the left hind limb. Ligations were made in the left femoral artery proximal to the superficial epigastric artery branch and anterior to the saphenous artery. Then, the femoral artery and the attached branches between ligations were excised. The skin was closed using a 4-0 suture line and erythromycin ointment was applied to prevent wound infection after surgery. Recovery of the blood flow was evaluated before and after surgery using a dynamic microcirculation imaging system (Teksqray, Shenzhen, China). Relative blood flow recovery is expressed as the ischemia to non-ischemia ratio. At least three mice were included in each experimental group.

Cell Culture

HEK293 cells and human umbilical vein endothelial cells (HUVECs) were purchased from ATCC. HEK293 cells were cultured in Gibco® DMEM (Life Technologies, USA) supplemented with 10% fetal bovine serum (FBS) at 37° C., 5% CO₂. HUVECs were cultured in Gibco® M199 (Life Technologies, USA) supplemented with 15% FBS, 50 μg/ml endothelial cell growth supplement (ECGS) and 100 μg/ml heparin at 37° C., 5% CO₂. All cell lines used were authenticated by short tandem repeat (STR) profile analysis and were mycoplasma free.

RNA Isolation and Quantitative PCR (Q-PCR) Analysis

Total RNA was extracted from cells or mouse tissues using Trizol® reagent RNAiso Plus (Takara, Japan) and transcribed into cDNA using 5× Primescript RT Master Mix (Takara, Japan), following the manufacturer's instructions. The mRNA levels were determined by quantitative PCR with SYBR Premix Ex Taq II (Takara, Japan) detected on a CFX Connected Real-Time PCR Detection System (Bio-Rad).

Protein Extraction and Western Blotting

For protein extraction, cells were suspended in SDS lysis buffer and boiled. Then, the lysate was centrifuged at 12,000×g for 2 min and the supernatant was collected. For Western blotting, protein samples were separated on SDS-polyacrylamide gels, transferred to PVDF membranes (Millipore, USA), blocked with 5% non-fat milk and incubated with the relevant antibodies. Images were acquired on a Bio-Rad system.

Immunofluorescence Staining

Frozen sections of aorta, skeletal muscle and liver tissues were fixed in 4% PFA, permeabilized with 0.3% Triton X-100, blocked with 5% BSA and 1% goat serum, and then incubated with primary antibodies at room temperature for 2 h or at 4° C. overnight. After three washes with PBST, the sections were incubated with secondary antibodies for 1 h at room temperature and then stained with DAPI anti-fade mounting medium. Images were captured under a Zeiss LSM880 confocal microscope.

Masson Trichrome Staining

Paraffin-embedded sections of PFA-fixed tissues were dewaxed and hydrated. Staining was then performed using a Masson trichrome staining kit (Beyotime, China). In brief, the sections were dipped in Bouin buffer for 2 h at 37° C., and then successively stained with Celestite blue staining solution, Hematoxylin staining solution, Ponceau's staining solution and Aniline blue solution for 3 minutes. After dehydrating with ethyl alcohol three times, the sections were mounted with Neutral Balsam Mounting Medium (BBI Life Science, China). Images were captured under a Zeiss LSM880 confocal microscope.

Fluorescence Activated Cell Sorting

Mice were sacrificed by decapitation. The lungs were then collected, cut into small pieces and then digested with collagenase I (200 U/ml) and neutral protease (0.565 mg/ml) for 1 h at 37° C. The isolated cells were incubated with PE-conjugated anti-CD31 antibody for 1 h at 4° C. and then 7-AAD (1:100) for 5 min. CD31-positive and 7-AAD-negative cells were sorted on a flow cytometer (BD biosciences, USA).

Myography

Four months old male mice were anesthetized with 4% chloral hydrate by intraperitoneal injection. Thoracic aortas were collected, rinsed in ice-cold Krebs solution and cut into 2 mm-length rings. Each aorta ring was bathed in 5 ml oxygenated (95% O₂ and 5% CO₂) Krebs solution at 37° C. for 30 min in a myograph chamber (620M, Danish Myo Technology). Each ring was stretched in a stepwise fashion to the optimal resting tension (thoracic aortas to ˜9 mN) and equilibrated for 30 min. Then, 100 mM K+ Krebs solution was added to the chambers to elicit a reference contraction and then washed out with Krebs solution at 37° C. until achieved a baseline. Vasodilatation induced by acetylcholine (Ach) or sodium nitroprusside (SNP) (1 nM to 100 μM) was recorded in 5-HT (2 μM) contracted rings. Data are represented as a percentage of force reduction and the peak of K+-induced contraction. At least three mice were included in each experimental group.

Mice/Human Cytokine Antibody Array

A cytokine assay for mice or human samples (RayBio®) was performed according to the manufacturer's instructions. Briefly, membranes were incubated in blocking buffer for 30 min at room temperature. The samples prepared from serum or cell lysates were added to each membrane and incubated for 4 h at room temperature. After three washes with buffer 1 and two washes with buffer 2, the membranes were reacted with a biotinylated antibody cocktail at 4° C. overnight. After incubation with 1000×HRP-Streptavidin for 2 h, the membranes were again washed three times with buffer 1 and two times with buffer 2 and then visualized using a Bio-Rad detection system. At least three mice were included in each experimental group.

Echocardiography

7-8 months old male mice were anesthetized by isoflurane gas inhalation and then subjected to transthoracic echocardiography (IU22, Philips). Parameters, including heart rate, cardiac output, left ventricular posterior wall dimension (LVPWD), left ventricular end-diastolic dimension (LVEDD), left ventricular end-systolic diameter (LVESD), LV ejection fraction and LV fractional shortening were acquired. At least three mice were included in each experimental group.

Bone Density Analysis

7-8 months old male mice were sacrificed by decapitation. The thigh bone was fixed in 4% PFA at 4° C. overnight. The relevant data were collected by micro-CT (Scanco Medical, μCT100). At least three mice were included in each experimental group.

Endurance Running Test

A Rota-Rod Treadmill (YLS-4C, Jinan Yiyan Scientific Research Company, China) was used to monitor fatigue resistance. Briefly, mice were placed on the rotating lane and the speed of the rotations gradually increased to 40 r/min. When the mice were exhausted, they were safely dropped from the rotating lane and the latency to fall was recorded. At least three mice were included in each experimental group.

10× Genomics Single-Cell-RNA-Sequencing

CD31⁺ cells isolated from murine lung by FACS (>90% viability) were used for single-cell RNA sequencing. A sequence library was built according to the Chromium Single Cell Instrument library protocol (49). Briefly, single-cell RNAs were barcoded and reverse-transcribed using the Chromium™ Single Cell 3′ Reagent Kits v2 (10× Genomics), then fragmented and amplified to generate cDNAs. The cDNAs were quantified using an Agilent Bioanalyzer 2100 DNA Chip, and the library was sequenced using an Illumina Hiseq PE150 with ˜10-30M raw data assigned for each cell. The reads were mapped to the mouse mm9 genome and analyzed using STAR: >90% reads mapped confidently to genomic regions and >50% mapped to exonic regions. Cell Ranger 2.1.0 was employed to align reads, generate feature-barcode matrices and perform clustering and gene expression analysis. >80,000 mean reads and 900 median genes per cell were obtained. The UMI (unique molecular identifier) counts were used to quantify the gene expression levels and the t-SNE algorithm was used for dimensionality reduction. The cell population was then clustered by k-means clustering (k=4). The Log 2FoldChange was the ratio of gene expression of one cluster to that of all other cells. The p-value was calculated using the negative binomial test and the false discovery rate was determined by Benjamini-Hochberg procedure. GO and KEGG enrichment analysis were performed in DAVID version 6.8. (50).

Statistical Analysis

A two-tailed Student's t-test was used to determine statistical significance, except that the statistical comparison of survival data was performed by Log-rank Test. All data are presented as the means±s.d. or means±s.e.m. as indicated, and a p value <0.05 was considered statistically significant.

### EXAMPLE 1

Single-Cell Transcriptomic Analysis Reveals Four Predominant Cell Clusters in CD31⁺ Murine Lung Endothelial Cells (MLECs)

To study the mechanism of VE aging, we generated a mouse model of conditional progerin knock-in, in which the Lmna^{G609G} mutation, equivalent to HGPS LMNA^{G608G}, was flanked with loxP sites, i.e. Lmna^{f/f} mice (FIG. 8A). The Lmna^{f/f} mice were crossed to E2A-Cre mice, in which the Cre recombinase is ubiquitously expressed including germ cells, to generate Lmna^{G609G/G609G} and Lmna^{G609G/+} mice. Progerin was ubiquitously expressed in Lmna^{G609G/G609G} and Lmna^{G609G/+} mice, which recapitulated many progeroid features found in HGPS, including growth retardation and shortened lifespan (FIGS. 8B-D).

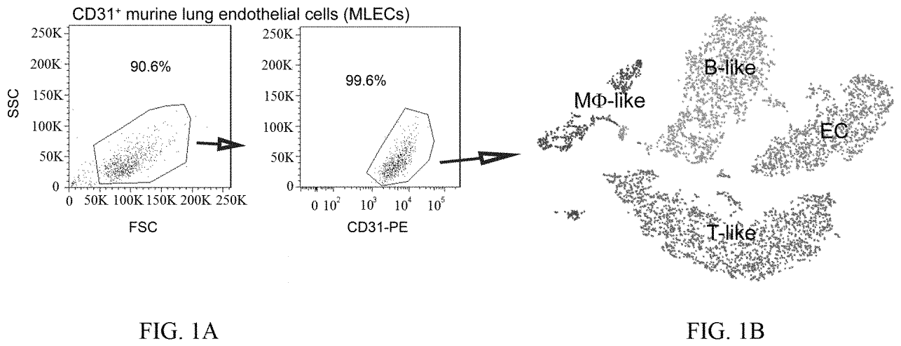

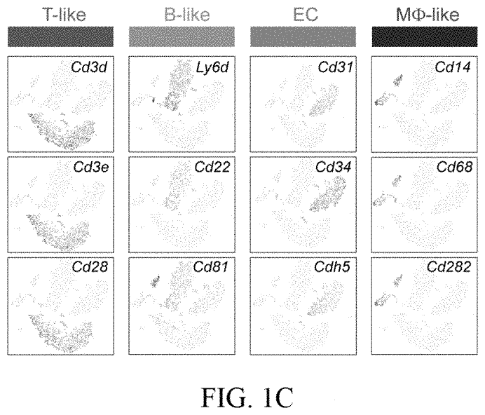

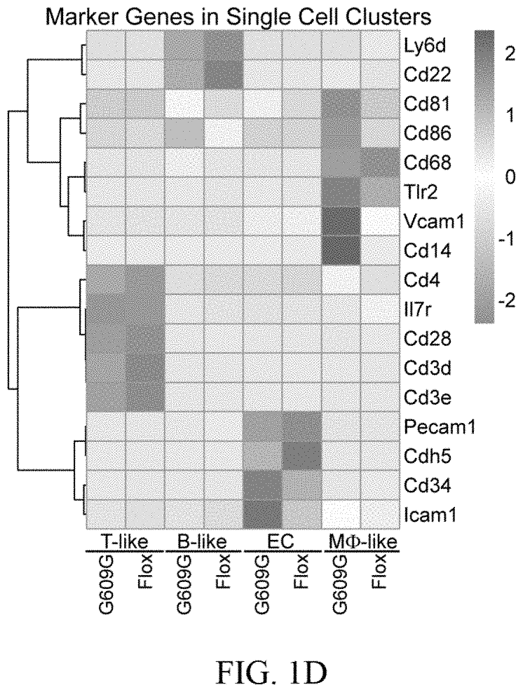

To understand primary alterations in the VE, we isolated CD31⁺ MLECs (25) from three pairs of Lmna^{G609G/G609G} (G609G) and Lmna^{f/f} (Flox) mice by FACS (FIG. 1A) and performed 10× Genomics single-cell RNA sequencing. We recovered 6,004 cells (4,137 from G609G and 1,867 from Flox mice) and used the k means clustering algorithm to cluster the cells into four groups (FIG. 1B). As expected, one group exhibited high Cd31, Cd34 and Cdh5 expression, and thus largely represented MLECs. The other three groups, co-purified with CD31⁺ MLECs by FACS, showed relatively lower Cd31 expression at mRNA level (>10-fold lower than MLECs) but high Cd45 expression (FIGS. 9A-B). Further analysis revealed that these clusters most likely contained B lymphocytes (B-like), with high Cd22, Cd81 and Ly6d expression; T lymphocytes (T-like) with high Cd3d, Cd3e and Cd28 expression; and Macrophages (Mφ-like) with high Cd14, Cd68 and Cd282 expression (FIG. 1C). Most of the marker-gene expression levels were comparable between G609G and Flox mice, except for Cd34 and Icam1, which were significantly elevated in G609G ECs, and Cd14 and Vcam1, which were increased in G609G Mϕ-like cells (FIG. 1D). Of note, Icam1 and Vcam1 are among the most conserved markers of endothelial senescence and atherosclerosis. Thus, we established a Lmna^{f/f} conditional progerin KI mouse model and revealed a unique EC population for mechanistic study.

### EXAMPLE 2

Progeroid ECs Develop a Systemic Inflammatory Response

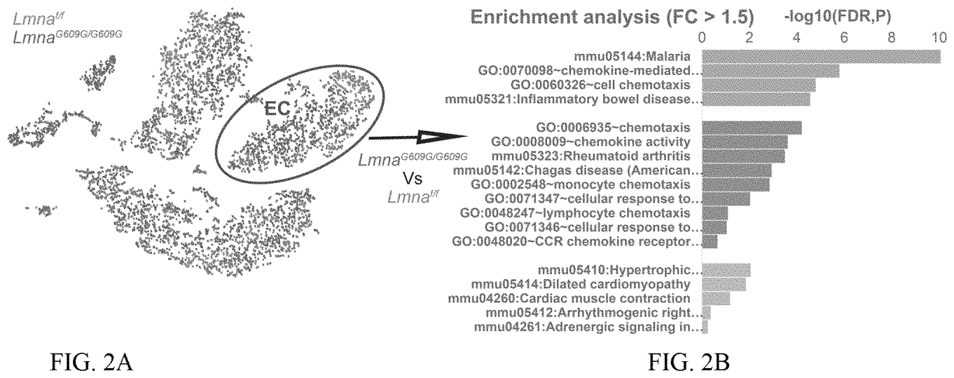

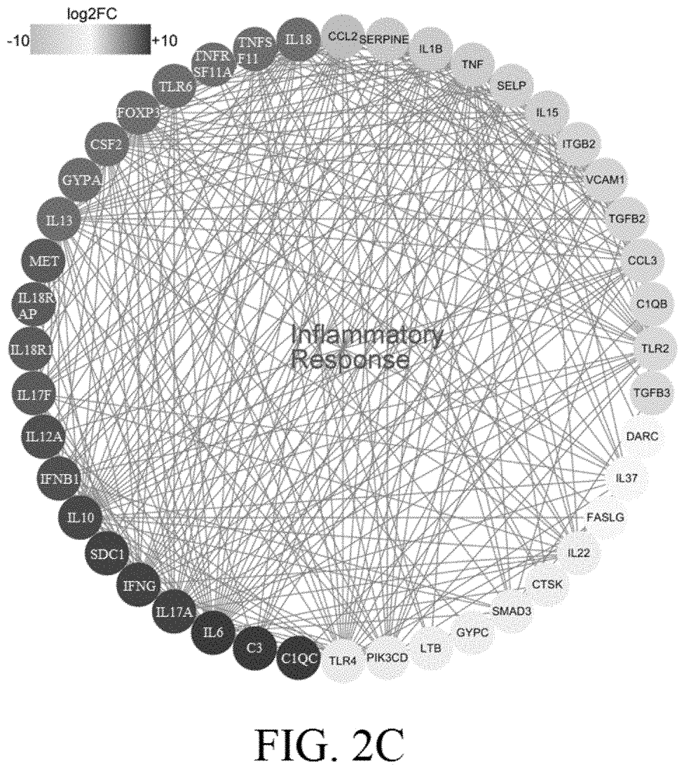

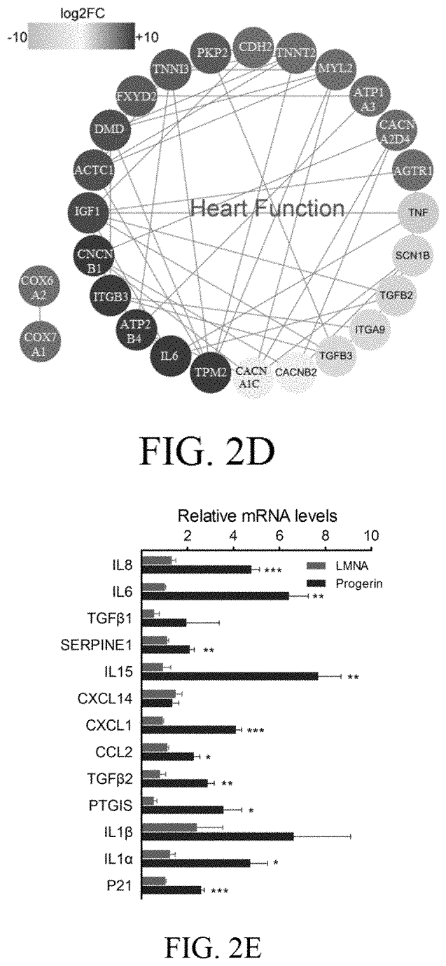

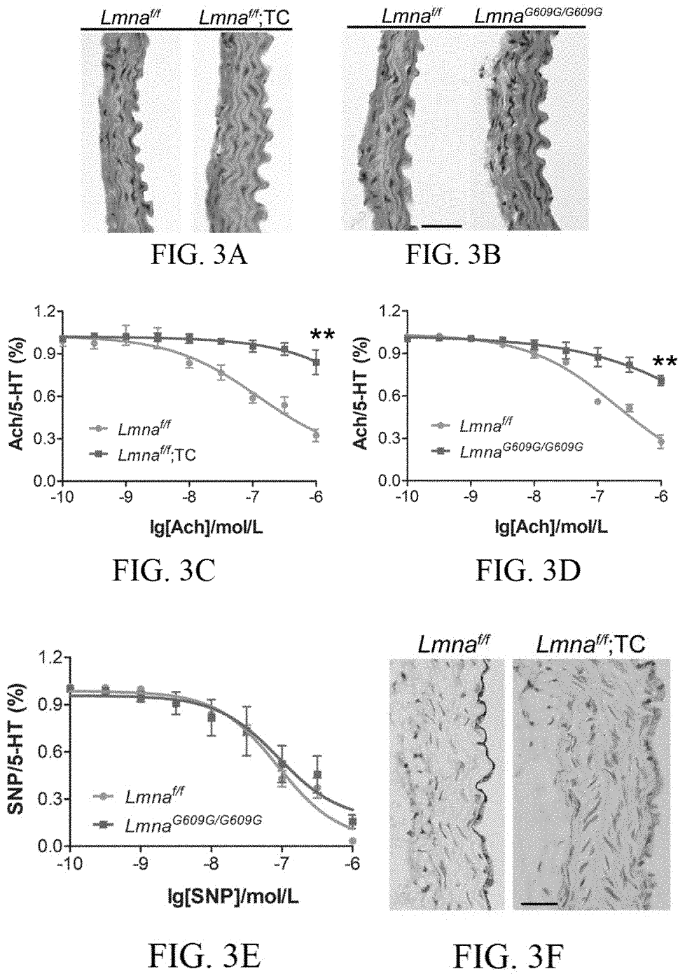

Of the four clusters of CD31⁺ MLECs, ECs and Mϕ-like cells showed high levels of p21^{CiP1/Waf1} (FIG. 9A), a typical senescence marker. This finding suggests that these cells are the main target of progerin in the context of aging. Interestingly, a previous study reported that Mφ-specific progerin, achieved by crossing Lmna^{f/+} to Lyz-Cre mice, caused minimal aging phenotypes (23), implicating that Mφ might have only a minor role in organismal aging. We thus focused on ECs for further analysis. We recovered 899 and 445 ECs from E2A and Flox mice, respectively (FIG. 2A). Genes with >1.5-fold change in expression between these mice were chosen for GO and KEGG analysis. We observed a significant enrichment in the pathways that regulate chemotaxis, immune responses in Malaria and Chagas diseases, inflammatory bowel disease and rheumatoid arthritis and pathways essential for cardiac function (FIGS. 2B-D). To confirm this observation, and to exclude paracrine effects from other cell types, we overexpressed progerin in human umbilical vein endothelial cells (HUVECs) and analyzed representative genes by quantitative PCR. Most of the examined genes, e.g. IL6, IL8, IL15, CXCL1 and IL1α etc. were significantly upregulated upon ectopic progerin overexpression (FIG. 2E). Together, these data suggest that progerin causes an inflammatory response in VE, which might lead to systemic aging.

### EXAMPLE 3

VE Dysfunction Causes Vasodilation Defects in Progeria Mice

To test whether the VE dysfunction has essential roles in systemic aging, we crossed Lmna^{f/f} mice to a Tie2-Cre line to generate Lmna^{f/f}; TC mice, in which the expression of Cre recombinase is driven by the promoter/enhancer of endothelial-specific Tie2 gene (26). Single-cell transcriptome analysis confirmed that Tie2 was mainly detected in ECs (FIG. 9B). Consistently, progerin was observed in the VE of Lmna^{f/f}; TC but not in that of Lmna^{f/f} control mice or other tissues (FIGS. 10A-B). VE-specific progerin induced intima-media thickening in Lmna^{f/f}; TC mice, in a similar manner as total KI mice, i.e. Lmna^{G609G/G609G} mice (FIGS. 4A-B). We performed functional analysis of the VE based on acetylcholine (ACh)-regulated vasodilation. ACh-induced thoracic aorta relaxation was significantly compromised in Lmna^{f/f}; TC mice (FIG. 4C). Similar defects were observed in Lmna^{G609G/G609G} and Lmna^{G609G/+} mice (FIGS. 4D and 11), where progerin was expressed in both ECs and SMCs (23). To gain more evidence supporting VE-specific dysfunction, we examined thoracic aorta relaxation induced by sodium nitroprusside (SNP), which is a SMC-dependent vasodilator. Little difference was observed in thoracic aorta vasodilation in Lmna^{G609G/G609G} and Lmna^{G609G/+} compared to Lmna^{f/f} control mice (FIG. 4E and FIG. 11), supporting that the VE dysfunction is a key contributor of vasodilation defects in progeria mice. As NO is the most potent vasodilator, we examined eNOS levels in thoracic aorta of Lmna^{f/f}; TC and Lmna^{f/f} control mice As expected, the level of eNOS was significantly reduced in Lmna^{f/f}; TC mice compared to Lmna^{f/f} control mice (FIG. 4F). Thus, the data confer a VE-specific dysfunction in progeria mice.

### EXAMPLE 4

Defective Neovascularization Following Ischemia in Progeria Mice

The reduced capillary density and neovascularization capacity are both characteristics of endothelial dysfunction (1). We examined the microvasculature in various tissues of Lmna^{f/f}; TC mice by immunofluorescence staining. We observed a significant loss in CD31⁺ ECs in Lmna^{f/f}; TC mice compared to controls (FIGS. 5A-B). We further examined ischemia-induced neovascularization ability in Lmna^{f/f}; TC mice following femoral artery ligation. Indeed, limb perfusion after ischemia was significantly blunted in Lmna^{f/f}; TC mice compared to controls (FIG. 5C). Histological analysis confirmed that the defect in blood-flow recovery in Lmna^{f/f}; TC mice was a reflection of an impaired ability to form new blood vessels in the ischemic region (FIG. 5D). Together, Lmna^{f/f}; TC mice are characterized by a loss of ECs, a reduced capillary density and defective neovascularization capacity.

### EXAMPLE 5

Endothelial Dysfunction is a Causal Factor of Systemic Aging

The single-cell transcriptome implicates heart dysfunction in Lmna^{G609G/G609G} mice (FIGS. 2A-E). A correlation with gene alterations associated with atherosclerosis and osteoporosis was obvious in Lmna^{G609G/G609G} ECs (the Online Mendelian Inheritance in Man database) (FIGS. 12A-B). We thus reasoned that endothelial-specific dysfunction might be enough to trigger systemic aging. Strikingly, atherosclerosis was prominent in Lmna^{f/f}; TC mice (FIG. 5A; aorta atheromatous plaque observed in all eight examined mice), as well as severe fibrosis in the arteries and hearts (FIGS. 5B-C); both are typical features of aging. Moreover, the heart/body weight ratio was significantly increased in Lmna^{f/f}; TC compared to Lmna^{f/f} control mice (FIG. 5D). Echocardiography confirmed that heart rate, cardiac output, left ventricular ejection fraction (LVEF) and fractional shortening (LVFS) were significantly reduced in 7-8-month old Lmna^{f/f}; TC compared to Lmna^{f/f} control mice. The running endurance was largely compromised in Lmna^{f/f}; TC mice (FIG. 5E), which is likely a reflection of amyotrophy and/or heart dysfunction. Moreover, the micro-computed tomography (micro-CT) identified a decrease in trabecular bone volume/tissue volume (BV/TV), trabecular thickness (Tb.Th) and trabecular number (Tb.N) but an increase of trabecular separation (Tb.Sp) in Lmna^{f/f}; TC mice (FIG. 5F), indicative of osteoporosis, which is an important hallmark of systemic aging (27). Most remarkably, the VE-specific dysfunction not only accelerated aging in various tissues/organs, but also shortened the median lifespan of Lmna^{f/f}; TC mice (24 weeks), to a similar extent as Lmna^{G609G/G609G} mice (21 weeks) (FIG. 5G). Interestingly, Lmna^{G609G/G609G} mice suffered from body-weight loss roughly from 8 weeks of age, while Lmna^{f/f}; TC mice only showed a slight drop in body weight (FIG. 5H), suggesting that body-weight loss itself is a less likely primary causal factor to progeria compared to endothelial dysfunction. Together, these results implicate that endothelial dysfunction, at least in progeria, acts as a causal factor of systemic aging.

### EXAMPLE 6

Accumulation of Progerin Destabilizes SIRT7

Loss of Sirt7, an NAD+-dependent deacylase, causes heart dysfunction with systemic inflammation and accelerates aging (28, 29). We noticed defective neovascularization in Sirt7 KO mice (FIG. 6A). Knockdown of Sirt7 upregulated the levels of IL1β and IL6 in HUVECs, as determined by Western blotting and real-time PCR (FIGS. 6B-C). Significantly, the protein level of Sirt7 was reduced almost 50% in Lmna^{f/f}; TC MLECs (FIG. 6D). By contrast, the levels of Sirt6 and Sirt1 were hardly decreased in Lmna^{f/f}; TC MLECs. Further, co-immunoprecipitation (Co-IP) revealed that lamin A interacted with Sirt7, which was significantly enhanced in case of progerin (FIG. 6E). HA-SIRT7 was poly-ubiquitinated, which was enhanced in presence of progerin compared with lamin A (FIG. 6F). Ectopic expression of progerin in HUVECs accelerated SIRT7 protein degradation, which was inhibited by MG132 (FIG. 6E). These data suggest that accumulation of progerin destabilizes Sirt7 by proteasomal pathway in progeria cells.

### EXAMPLE 7

VE-Specific Expression of Sirt7 Ameliorates Aging Features and Extends Lifespan

We reasoned that Sirt7 might underlie the VE dysfunction in progeria mice. To test this hypothesis, we first examined whether ectopic Sirt7 could rescue the exacerbated inflammatory response in HUVECs. As shown, overexpression of Sirt7 significantly downregulated the expression of multiple inflammatory genes like IL1β (FIG. 7A). To test the in vivo function of Sirt7 in defective neovascularization, we generated a recombinant AAV serotype 1 (rAAV1) cassette with Sirt7 gene expression driven by a synthetic ICAM2 promoter (IS7O), which ensures VE-specific expression (30, 31). As shown, on-site injection of IS7O at a dose of 1.25×10¹⁰ viral genome-containing particles (vg)/50 μl significantly improved blood vessel formation in Lmna^{f/f}; TC mice (FIG. 7B). The ectopic expression of Sirt7 and increase of CD31-labeled ECs was evidenced by fluorescence confocal microscopy in ECs of regenerated blood vessels (FIGS. 7C-D).

We next asked whether IS7O could ameliorate premature aging and extend lifespan. To this end, the IS7O particles were injected via tail vein from 21 weeks of age, when progeria mice start to die. The injection was repeated every other week at a concentration of 5×10¹⁰ vg/200 μl/mouse. While all untreated mice died before 34 weeks of age, most 5 IS7O-treated mice were still alive at the age of 44 weeks, when they were sacrificed for histological analysis. The ectopic expression of FLAG-SIRT7 was observed in the ECs of liver, muscle and aorta but not in bone marrow cells (WBMCs), determined by fluorescence microscopy and/or Western blotting (FIGS. 7E-F). Most remarkably, the median lifespan was extended by 76%—from 25 to >44 weeks (FIG. 7G). The age-related body-weight loss was slightly rescued upon IS7O therapy in Lmna^{f/f}; TC mice (FIG. 7H). These data suggest that progerin-caused VE dysfunction and systemic aging are partially, if not entirely, attributable to Sirt7 decline.

### DISCUSSION

Mounting evidence supports that endothelial dysfunction is a conspicuous marker for vascular aging and CVDs (32-34). However, fundamental question remains whether VE dysfunction causally triggers systemic aging. The heterogeneity of vascular cells and their close communication with the blood stream renders it difficult to understand the primary function of the VE. The murine Lmna^{G609G} mutation, equivalent to the LMNA^{G608G} found in humans with HGPS, causes premature aging phenotypes in various tissues/organs, thus providing an ideal model for studying aging mechanisms at both tissue and organismal levels. Data from Lmna^{G609G} model suggest that SMCs are the primary cause of vascular diseases, such as atherosclerosis (17-22). A recent study showed that specific expression of Lmna^{G609G} in SMCs causes atherosclerosis and shortens lifespan in atherosclerosis-prone Apoe^{-/-} mice (23). We used Tie2-Cre line to generate VE-specific Lmna^{G609G} mouse model. The Lmna^{f/f}; TC mice exhibited vascular dysfunction, accelerated aging and a shortened lifespan to a similar extent to the whole body Lmna^{G609G} model. Tie2 expression was reported not only in ECs but also in hematopoietic lineages (35). Our single-cell transcriptomic data identified Tie2 transcripts mainly in MLECs instead of B-, T- or Mφ-like cells. When a synthetic ICAM2 promoter was employed to drive ectopic expression of FLAG-SIRT7 in the rescue experiments, ectopic FLAG-SIRT7 was successfully detected in ECs of aorta, muscle and liver, but hardly detected in WBMCs. Therefore, Tie2-driven progerin expression combined with synthetic ICAM2-drivern SIRT7 rescue largely ensure the EC specific contribution in systemic aging. Of note, although the number and function of HSCs decline in another progeria model, Zmpste24^{-/-} mice (15), little effect was observed when healthy hematopoietic progenitor cells was transplanted to Zmpste24^{-/-} mice in the context of systemic aging. Recently, Hamczyk et al. found that knocking in the Lmna^{G609G} allele in macrophages mediated by LysM-Cre merely affects aging and lifespan (23). Therefore our data strongly suggest that, as the largest secretory organ (3, 4), VE is pivotal in regulating systemic aging and longevity. In support of our findings, Foisner et al. reported that VE-cadherin promoter-driven expression of progerin in a transgenic line causes cardiovascular abnormalities and shortens lifespan (36).

One limitation in the understanding of mechanisms of VE dysfunction is the vascular cell heterogeneity and the lack of appropriate in vitro system for ECs. Here, we took advantage of single-cell RNA sequencing technique to analyze the transcriptomes of MLECs. Surprisingly, although >95% purity was achieved by FACS, MLECs isolated by CD31-immunofluorescence labeling turned out to be a mixture of cells, including ECs, T-like, B-like and Mϕ-like cells. Though enriched by FACS, these non-EC cells expressed low level of CD31 mRNA, raising the possibility that cell surface proteins like CD31 T-like, B-like and Mϕ-like cells might be obtained from neighbor ECs via intercellular protein transfer (37). Nevertheless, these findings suggest that one can't just purify CD31⁺ cells and pool them together for mechanistic study, otherwise might get misleading conclusion. We compared the expression of genes that are associated with atherosclerosis, arthritis, heart failure, osteoporosis or amyotrophy (the Online Mendelian Inheritance in Man database) between progeroid and control in all four clusters. An obvious alteration of these genes/pathway was observed mainly in ECs and Mϕ-like cells. At current stage, it is hard to separate cell-autonomous and paracrine effects among different cell populations. In future, it would be worth to do an analysis in Lmna^{f/f}; TC MLECs. The data will be useful to study the paracrine effect of ECs on other cell populations.

Since the identification of the causal link between LMNA G608G mutation and HGPS, numerous efforts have been being put on the developing of treatment for HGPS. FTIs (39, 40), resveratrol and N-acetyl cysteine (NAC) (15) treatment alleviate premature aging features and extend lifespan in progeria murine models. Rapamycin (41) and metformin (42) incubation rescue senescence in HGPS cells. Based on these notions, HGPS patients taking FTI-lonafarnib in clinical trial showed significant improvement of health status, reduction of mortality rate and a potential extension of lifespan (about 1-2 years) (43-45). Taking advantages of gene therapy and the dispensable role of lamin A, morpholino oligos (16) and CRISPR—Cas9 designs (46, 47), which prevent lamin A/progerin generation, can alleviate aging features and extend lifespan from 25% to 40% in progeria mice. However, considering the indispensable function of lamin A in humans, these genome-modifying strategies need further experimentation before potential clinical application. Here, applying different strategy, we showed that rAAV1-SIRT7 (IS7O), targeting dysfunctional VE, could largely ameliorate progeroid features and almost double the median lifespan (from 25 weeks to >44 weeks). To our best knowledge, this is the most dramatic rescue of progeria in mouse model via gene therapy.

Collectively, we reveal VE dysfunction as a primary trigger of systemic aging and as a risk factor for age-related diseases like atherosclerosis, heart failure and osteoporosis. Drugs and molecules that target VE might serve as good candidates in the treatment of age-related diseases other than CVDs. The findings in SIRT7-based gene therapy implicate great clinical potentials for progeria as well as in anti-aging applications.

### REFERENCES

1. D. G. Le Couteur, E. G. Lakatta, A vascular theory of aging. J Gerontol A Blot Sci Med Sci 65, 1025-1027 (2010).
2. X. L. Tian, Y. Li, Endothelial cell senescence and age-related vascular diseases. J Genet Genomics 41, 485-495 (2014).
3. H. A. Hadi, C. S. Carr, J. Al Suwaidi, Endothelial dysfunction: cardiovascular risk factors, therapy, and outcome. Vasc Health Risk Manag 1, 183-198 (2005).
4. R. P. Brandes, I. Fleming, R. Busse, Endothelial aging. Cardiovasc Res 66, 286-294 (2005).
5. Y. T. Ghebre et al., Vascular Aging: Implications for Cardiovascular Disease and Therapy. Transl Med (Sunnyvale) 6, (2016).
6. J. Tao et al., Reduced arterial elasticity is associated with endothelial dysfunction in persons of advancing age: comparative study of noninvasive pulse wave analysis and laser Doppler blood flow measurement. Am J Hypertens 17, 654-659 (2004).
7. L. J. Ignarro et al., Role of the arginine-nitric oxide pathway in the regulation of vascular smooth muscle cell proliferation. Proc Natl Acad Sci USA 98, 4202-4208 (2001).
8. W. S. Cheang et al., Metformin protects endothelial function in diet-induced obese mice by inhibition of endoplasmic reticulum stress through 5′ adenosine monophosphate-activated protein kinase-peroxisome proliferator-activated receptor delta pathway. Arterioscler Thromb Vasc Biol 34, 830-836 (2014).
9. X. Li, Y. Dai, T. Shen, C. Gao, Induced migration of endothelial cells into 3D scaffolds by chemoattractants secreted by pro-inflammatory macrophages in situ. Regen Biomater 4, 139-148 (2017).
10. P. Scaffidi, T. Misteli, Lamin A-dependent nuclear defects in human aging. Science 312, 1059-1063 (2006).
11. D. McClintock et al., The mutant form of lamin A that causes Hutchinson-Gilford progeria is a biomarker of cellular aging in human skin. PloS one 2, e1269 (2007).
12. K. Cao et al., Progerin and telomere dysfunction collaborate to trigger cellular senescence in normal human fibroblasts. J Clin Invest 121, 2833-2844 (2011).
13. A. De Sandre-Giovannoli et al., Lamin a truncation in Hutchinson-Gilford progeria. Science 300, 2055 (2003).
14. M. Eriksson et al., Recurrent de novo point mutations in lamin A cause Hutchinson-Gilford progeria syndrome. Nature 423, 293-298 (2003).
15. B. Liu et al., Resveratrol Rescues SIRT1-Dependent Adult Stem Cell Decline and Alleviates Progeroid Features in Laminopathy-Based Progeria. Cell metabolism 16, 738-750 (2012).
16. F. G. Osorio et al., Splicing-directed therapy in a new mouse model of human accelerated aging. Sci Transl Med 3, 106ra107 (2011).
17. R. Varga et al., Progressive vascular smooth muscle cell defects in a mouse model of Hutchinson-Gilford progeria syndrome. Proceedings of the National Academy of Sciences of the United States of America 103, 3250-3255 (2006).
18. D. McClintock, L. B. Gordon, K. Djabali, Hutchinson-Gilford progeria mutant lamin A primarily targets human vascular cells as detected by an anti-Lamin A G608G antibody. Proceedings of the National Academy of Sciences of the United States of America 103, 2154-2159 (2006).
19. G. H. Liu et al., Recapitulation of premature ageing with iPSCs from Hutchinson-Gilford progeria syndrome. Nature 472, 221-225 (2011).
20. J. Zhang et al., A human iPSC model of Hutchinson Gilford Progeria reveals vascular smooth muscle and mesenchymal stem cell defects. Cell Stem Cell 8, 31-45 (2011).
21. C. D. Ragnauth et al., Prelamin A acts to accelerate smooth muscle cell senescence and is a novel biomarker of human vascular aging. Circulation 121, 2200-2210 (2010).
22. Y Liu, I. Drozdov, R. Shroff, L. E. Beltran, C. M. Shanahan, Prelamin A accelerates vascular calcification via activation of the DNA damage response and senescence-associated secretory phenotype in vascular smooth muscle cells. Circulation research 112, e99-109 (2013).
23. M. R. Hamczyk et al., Vascular Smooth Muscle-Specific Progerin Expression Accelerates Atherosclerosis and Death in a Mouse Model of Hutchinson-Gilford Progeria Syndrome. Circulation 138, 266-282 (2018).
24. P. H. Kim et al., Disrupting the LINC complex in smooth muscle cells reduces aortic disease in a mouse model of Hutchinson-Gilford progeria syndrome. Sci Transl Med 10, (2018).
25. A. Longchamp et al., Amino Acid Restriction Triggers Angiogenesis via GCN2/ATF4 Regulation of VEGF and H2S Production. Cell 173, 117-129 e114 (2018).
26. Y Y. Kisanuki et al., Tie2-Cre transgenic mice: a new model for endothelial cell-lineage analysis in vivo. Dev Biol 230, 230-242 (2001).
27. H. Chen, X. Zhou, H. Fujita, M. Onozuka, K. Y. Kubo, Age-related changes in trabecular and cortical bone microstructure. Int J Endocrinol 2013, 213234 (2013).
28. B. N. Vazquez et al., SIRT7 promotes genome integrity and modulates non-homologous end joining DNA repair. EMBO J 35, 1488-1503 (2016).
29. S. Araki et al., Sirt7 Contributes to Myocardial Tissue Repair by Maintaining Transforming Growth Factor-beta Signaling Pathway. Circulation 132, 1081-1093 (2015).
30. C. Dai, R. E. McAninch, R. E. Sutton, Identification of synthetic endothelial cell-specific promoters by use of a high-throughput screen. J Virol 78, 6209-6221 (2004).
31. X. Huang et al., Genome editing abrogates angiogenesis in vivo. Nat Commun 8, 112 (2017).
32. H. Liu et al., Regulation of miR-92a on vascular endothelial aging via mediating Nrf2-KEAP1-ARE signal pathway. Eur Rev Med Pharmacol Sci 21, 2734-2742 (2017).
33. G. Cui et al., Lack of causal relationship between leukocyte telomere length and coronary heart disease. Atherosclerosis 233, 375-380 (2014).
34. A. de la Sierra, M. Larrousse, Endothelial dysfunction is associated with increased levels of biomarkers in essential hypertension. J Hum Hypertens 24, 373-379 (2010).
35. B. Kilani et al., Comparison of endothelial promoter efficiency and specificity in mice reveals a subset of Pdgfb-positive hematopoietic cells. J Thromb Haemost 17, 827-840 (2019).
36. S. Osmanagic-Myers et al., Endothelial progerin expression causes cardiovascular pathology through an impaired mechanoresponse. J Clin Invest, (2018).
37. K. A. Ahmed, J. Xiang, Mechanisms of cellular communication through intercellular protein transfer. J Cell Mol Med 15, 1458-1473 (2011).
38. A. Bantikassegn, X. Song, K. Politi, Isolation of epithelial, endothelial, and immune cells from lungs of transgenic mice with oncogene-induced lung adenocarcinomas. Am J Respir Cell Mol Biol 52, 409-417 (2015).
39. L. G. Fong et al., A protein farnesyltransferase inhibitor ameliorates disease in a mouse model of progeria. Science 311, 1621-1623 (2006).
40. S. H. Yang et al., A farnesyltransferase inhibitor improves disease phenotypes in mice with a Hutchinson-Gilford progeria syndrome mutation. The Journal of clinical investigation 116, 2115-2121 (2006).
41. K. Cao et al., Rapamycin reverses cellular phenotypes and enhances mutant protein clearance in Hutchinson-Gilford progeria syndrome cells. Sci Transl Med 3, 89ra58 (2011).
42. A. L. Egesipe et al., Metformin decreases progerin expression and alleviates pathological defects of Hutchinson-Gilford progeria syndrome cells. NPJ Aging Mech Dis 2, 16026 (2016).
43. L. B. Gordon et al., Clinical trial of a farnesyltransferase inhibitor in children with Hutchinson-Gilford progeria syndrome. Proceedings of the National Academy of Sciences of the United States of America 109, 16666-16671 (2012).
44. L. B. Gordon et al., Impact of farnesylation inhibitors on survival in Hutchinson-Gilford progeria syndrome. Circulation 130, 27-34 (2014).
45. L. B. Gordon et al., Association of Lonafarnib Treatment vs No Treatment With Mortality Rate in Patients With Hutchinson-Gilford Progeria Syndrome. JAMA 319, 1687-1695 (2018).
46. O. Santiago-Fernandez et al., Development of a CRISPR/Cas9-based therapy for Hutchinson-Gilford progeria syndrome. Nat Med 25, 423-426 (2019).
47. E. Beyret et al., Single-dose CRISPR-Cas9 therapy extends lifespan of mice with Hutchinson-Gilford progeria syndrome. Nat Med 25, 419-422 (2019).
48. A. Limbourg et al., Evaluation of postnatal arteriogenesis and angiogenesis in a mouse model of hind-limb ischemia. Nat Protoc 4, 1737-1746 (2009).
49. J. T. Neal et al., Organoid Modeling of the Tumor Immune Microenvironment. Cell 175, 1972-1988 e1916 (2018).
50. W. Huang da, B. T. Sherman, R. A. Lempicki, Systematic and integrative analysis of large gene lists using DAVID bioinformatics resources. Nat Protoc 4, 44-57 (2009).

1. An agent that enables Sirt7 gene expression.

2. The agent according to claim 1, wherein the agent is a vector.

3. The agent according to claim 2, wherein the vector is a plasmid, and/or a virus vector.

4. The agent according to claim 3, wherein the virus vector is a recombinant adeno-associated virus.

5. The agent according to claim 4, wherein the Sirt7 gene expression is vascular endothelium specific expression.

6. A method for manufacturing a medicament for improving neovascularization, ameliorating aging features, preventing aging, extending lifespan, and/or treating Hutchinson-Gilford progeria syndrome and/or age-related diseases, the method comprising using the agent according to claim 1.

7. The method according to claim 6, wherein the aging, HGPS, and/or age-related diseases are characterized by vascular endothelium dysfunction.

8. The method according to claim 6, wherein the age-related diseases are cardiovascular diseases, arthritis, amyotrophy, and/or osteoporosis.

9. The method according to claim 8, wherein the cardiovascular diseases are heart failure and/or atherosclerosis.

10. A method for improving neovascularization, ameliorating aging features, preventing aging, extending lifespan, and/or treating Hutchinson-Gilford progeria syndrome and/or age-related diseases, comprising administering a pharmaceutically effective amount of the agent according to claim 1 to a subject in need thereof.

11. The agent according to claim 4, wherein the virus vector is a recombinant adeno-associated virus serotype 1.

12. The agent according to claim 5, wherein the Sirt7 gene expression is driven by an ICAM2 promoter.
